(19) United States
(12) Patent Application Publication
(10) Pub. No.: US 2015/0001160 A1
(43) Pub. Date: Jan. 1, 2015

(54) SEPARATION OF BIOCOMPONENTS FROM DDGS

(71) Applicant: Aicardo Roa-Espinosa, Madison, WI (US)

(72) Inventor: Aicardo Roa-Espinosa, Madison, WI (US)

(21) Appl. No.: 14/190,332

(22) Filed: Feb. 26, 2014

Related U.S. Application Data
(63) Continuation-in-part of application No. 13/929,618, filed on Jun. 27, 2013.

(51) Int. Cl. C12F 3/00; C11B 13/00; C07K 1/14; C08B 1/00

(52) U.S. Cl. CPC C12F 3/00; C08B 1/00; C11B 13/00; C07K 1/14
USPC 210/705; 210/806; 210/727; 210/726

(57) ABSTRACT

A multi stage process for the progressive removal of protein and isolating streams containing cellulose fibers and oil from a waste stream containing Dried Distillers Grains with Solubles is disclosed. Targeted polymers are added to the source and separated streams prior to passing the streams through separation equipment including a rotary screen, a multi disk press, a dissolved air floatation device and optionally a centrifuge in which the waste stream is separated into a stream containing predominantly protein, a stream containing predominantly oil and a stream that contains predominantly cellulose and hemicellulose fibers.

### RELATED APPLICATIONS

This application is a continuation in part application claiming priority from non-provisional application Ser. No. 13/929,618 filed on Jun. 27, 2013.

### FIELD OF THE INVENTION

The present invention relates generally to a process of recovering useful materials from waste sources that include Dried Distillers Grains with Solubles also known by the acronym DDGS, waste materials from ethanol production and animal feed waste.

### BACKGROUND OF THE INVENTION

Thin stillage and distillers' grains are byproducts remaining after alcohol distillation from a fermented cereal grain mash. Both byproducts are used as energy and protein sources for ruminants. There are two main sources of these byproducts. The traditional sources were from brewers. However, more recently, ethanol plants such as corn, sugar cane, cassava and potatoes have become a growing source.

DDGS contain valuable bio-materials mainly fibers, oil and protein. The oil in DDGS could be used either as cooking oil or as a biofuel. The main protein in corn is Zein which has been used in the manufacture of a wide variety of commercial products, including coatings for paper cups, soda bottle cap linings, clothing fabric, buttons, adhesives, coatings and binders, recently this protein has been used as a coating for candy, nuts, fruit, pills, and other encapsulated foods and drugs. Additionally Zein can be further processed into resins and other bioplastic polymers. Fibers may be used as raw materials in the production of lignocellulosic ethanol. Residue materials from ethanol production contain fibers from which ethanol has been extracted. However, only about 50-70% of the ethanol in these materials is typically extracted leaving substantial portion of ethanol that is available for further extraction. Tables 1 and 2 provide a typical content breakdown of the various materials in DDGS.

TABLE 1
Cellulosic biomass
compositional analysis of DDGS.

| | Average |
|---|---|
| Dry matter | 88.8 |
| Water extractives | 24.7 |
| Ether extractives | 11.6 |
| Crude protein | 24.9 |
| Glucan (total) | 21.2 |
| Cellulose | 16 |
| Starch | 5.2 |
| Xylan and Arabinan | 13.5 |
| Xylan | 8.2 |
| Arabinan | 5.3 |
| Ash | 4.5 |
| Total dry matter | 100.4 |

TABLE 2
Nutritional Compositional analysis of DDGS.
Nutritional Compositional analysis

| | |
|---|---|
| Dry matter | 88.9 |
| Crude protein | 27.3 |
| Crude fat | 14.5 |
| Carbohydrates | 53.5 |
| Ash | 4.7 |
| Total | 100 |

It would therefore be desirable to provide a process to separate these materials in order to maximize their uses.

### SUMMARY OF THE PRESENT INVENTION

In an aspect of the present invention, a multi-stage substantially continuous process for separating a source stream intermixedly containing fibers, proteins and oil, the process being configured for separating the source stream into three streams each containing predominantly one component, the source stream containing Dried Distillers Grains with Solubles, the process comprises the stages of: providing a first stream comprising dried distillers grain with solubles, the dried distillers grain stream containing water, oil, protein and fibers, the fibers containing hemicellulose and cellulose components; separating a stream comprising predominantly proteins and a mix of oil in a water that form an stream comprising predominantly fiber materials from the source stream; separating a stream containing predominantly oil and a stream containing predominantly proteins from the water with oil; and progressively concentrating the stream containing predominantly proteins in at least one additional step.

These and other features, aspects and advantages of the present invention will become better understood with reference to the following drawings, description and claims.

### BRIEF DESCRIPTION OF THE DRAWINGS

FIG. 1 is a flow chart schematic of the process according to an embodiment of the present invention;

FIG. 2 is a flow chart schematic of the process according to another embodiment of the present invention, and

FIG. 3 is a flow chart schematic of the process according to yet another embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention.

Raw Dried Distillers Grains with Solubles (DDGS) typically contain water in the range of between about 85% to about 95%, but could also be higher or lower depending on the source. Targeted polymers are added to the process to accomplish two objectives:

a. Detach the oil molecules from the fibers. The oil is held in the open pores of the fibers and also tends to have a strong affinity to the fiber surfaces.

b. Separate water and solids from a generally a mixture of oils fats and protein. Depending on the composition of the mixture, this may require several steps wherein the protein is progressively removed from the mixture.

c. Separate four streams from the source stream containing DDGS: 1) a stream containing predominantly oil, 2) a stream containing predominantly cellulose and hemicellulose fibers, 3) a stream containing predominantly protein and 4) an effluent stream of predominantly water that may be discarded and, as such, must comply with COD and BOD regulations. In this context, a predominant content of a component means at least 75% by weight of the component in the stream.

The targeted polymers selected to aid in stream separation possess colloidal properties that make them conducive for components of the stream to agglomerate around these polymers. The process of the present invention comprises of two distinctly different embodiments depending on the pH of the DDGS which is largely a function of the source and the treatment the DDGS undergoes prior undergoing the process of the present invention. For DDGS having a pH higher than about 5, the chemical additives required for effective separation are different than those required for DDGS having a pH below about 5. These are described below in more detail, but the underlying processes and the end results they intend to accomplish are the same.

A. Separating Fibers From a Water Mixture of Protein and Oil From the DDGS Source Stream

In this stage, the DDGS source stream is treated sequentially with two polymers or a silicate depending on the pH and passed through a rotary screen. This produces 1) a relatively high solids stream rich in fibers stream that may contain small amounts of proteins and oil and 2) and a stream containing low non-aqueous fluids that is a mixture containing protein and oil. The low solids stream may contain from about 0.5 percent to about 5% solids. The high solids stream may contain about 25% to about 35% solids with the liquid portion comprising predominantly of water and smaller amounts (typically less than 15%) of protein. This fraction is expected to be substantially oil free.

The high solids stream may undergo a second protein recovery step. A multi disk press further removes a mixture of protein and concentrates the high solids fiber fraction to a range between about 40% to about 50% solids.

The fibers may undergo further treatments as will be described below.

B. Recovery of the Oil and the Protein From the Water a Mixture Containing the Protein and the Oil

In this phase, the oil is recovered in a clarifier aided by a polymeric addition to the mixture and optionally an oil skimmer that removes the oil that rises to the top of the clarifier. The protein and water is passed through a Dissolved Air Floatation Device (DAF) with micronized air where further separation of oil and protein takes place. A multi disk press concentrates the protein fraction from the DAF and removes water effluent that is substantially oil free.

The protein fraction streams originating from the multi stage removal steps may be combined into one stream.

C. Fiber Treatments

The fibers are treated in a pin macerator to prep them for further biofuel production as will be described below.

The polymers used in the process of the current invention selectively steer the protein and oil components to the low solids streams containing mostly water.

The following represents the important characteristics of these polymers used in the process.

### Polyamines

Molecular weight between 10,000 and 1,000,000.
Liquid form with 40 to 50% concentration.
Cationic site on the main chain.
Viscosity at 50% concentration of between 40 and 20,000 centipoises.
Any polyamine having two H₂N groups may be used in this application. An example may be 1,3-diaminopropane.

### Polydicyandiamide

Molecular weight: 3000 to 150,000.
Cationic sites on a side chain.
Liquid at 40 to 60% concentration.
Highly cationic.
Viscosity of the liquids: 50 to 300 centipoises.

Polydicyandiamide is obtained from the reaction of Dicyandiamide monomer and formaldehyde as shown below:

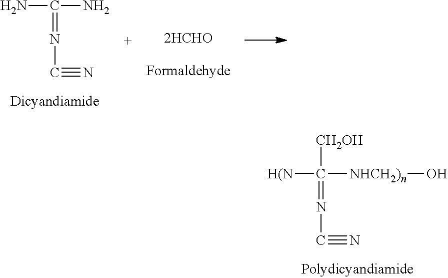

### Cationic Acrylamide Copolymers

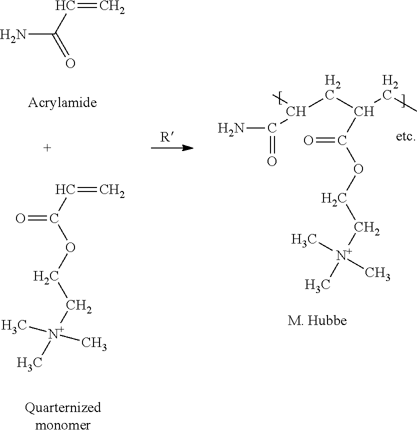

### ADMAEA

Acrylamide-dimethylaminoethyl acrylate copolymers.
The copolymerization of DMAEA-MeCl with acrylamide produces the cationic polymer.
The main characteristics of the products obtained are: Molecular weight: about 3 million to about 10 million.
Viscosity at 5 g/l: 100 to 1700 cps.
Specifically: acrylamide/Ethanaminium, N,N,N-trimethyl-2-((1-oxo-2-propenyl)oxo)-, chloride copolymer is a useful form of ADMAEA in the present invention.
The molecular formula is C₁₁H₂₁ClN₂O₃. The molecular structure is shown below in 2D.

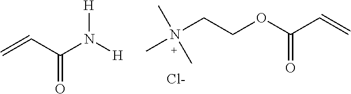

### Sodium or Potassium Anionic Acrylate Acrylamide Copolymer

This polymer may be made from the reaction between an acrylamide monomer and an acrylic acid monomer as shown below.

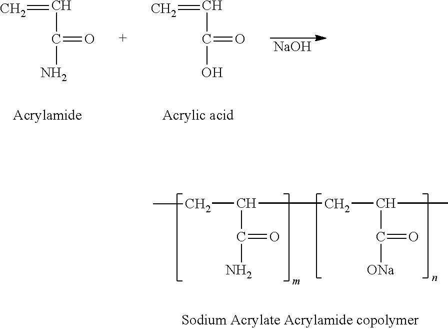

The anionicity of these copolymers can vary between 0% and 100% depending on the ratio of the monomers involved. The anionic copolymers used in the process of the present invention may have a molecular weight ranging between about 3 million to about 30 million, and a viscosity at a concentration of 5 g/l ranging from about 200 centipoises to about 2800 centipoises. The preferred pH range for making these copolymers is from 4.5 to 9. It is also noted that potassium may be substituted for the sodium as the base in the Acrylate Acrylamide copolymer.

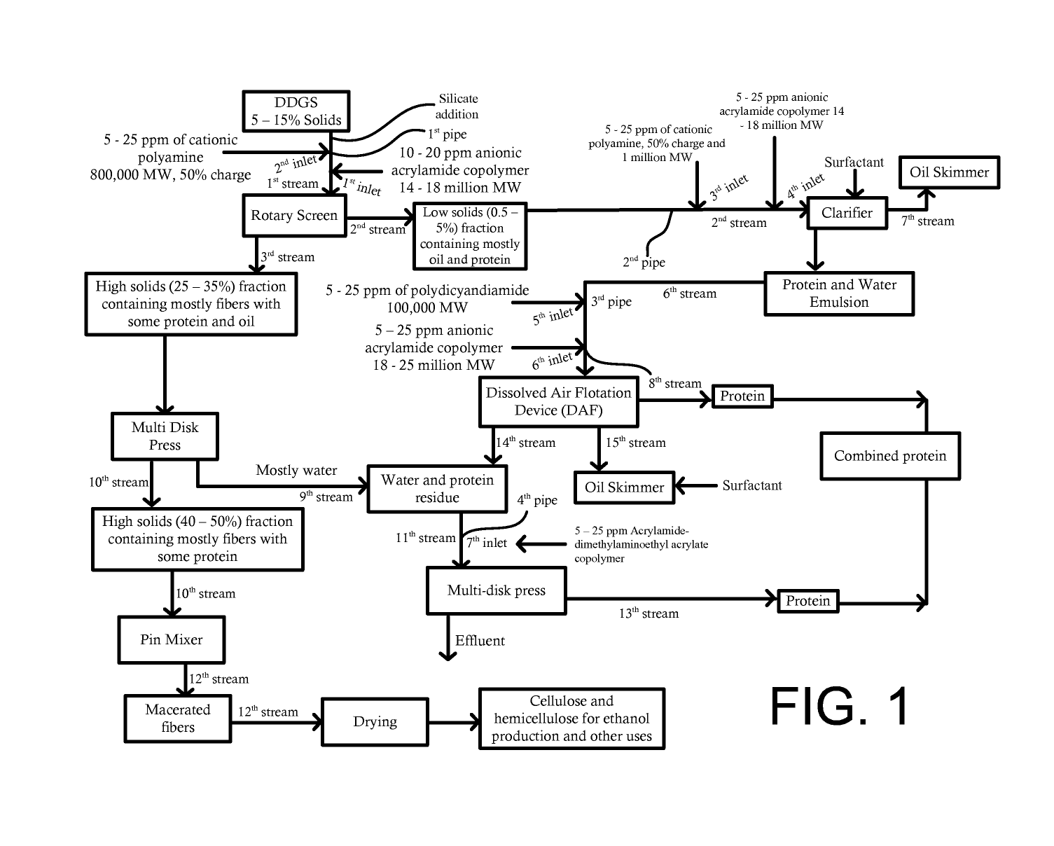

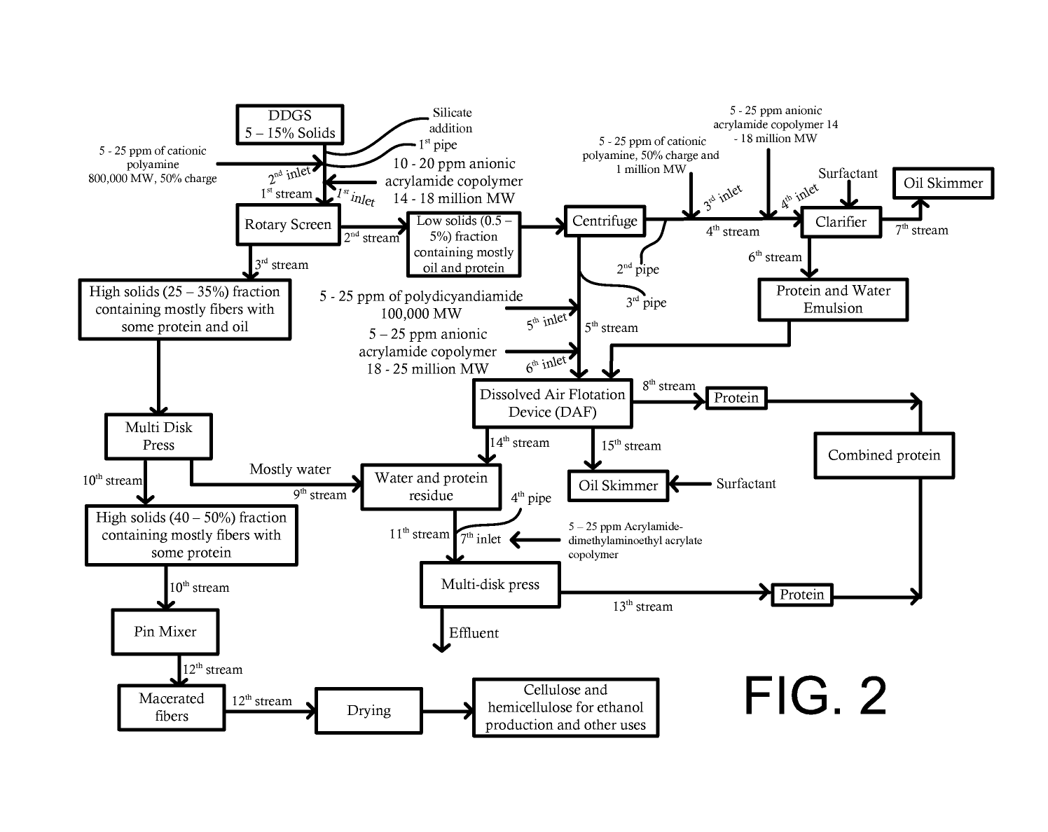

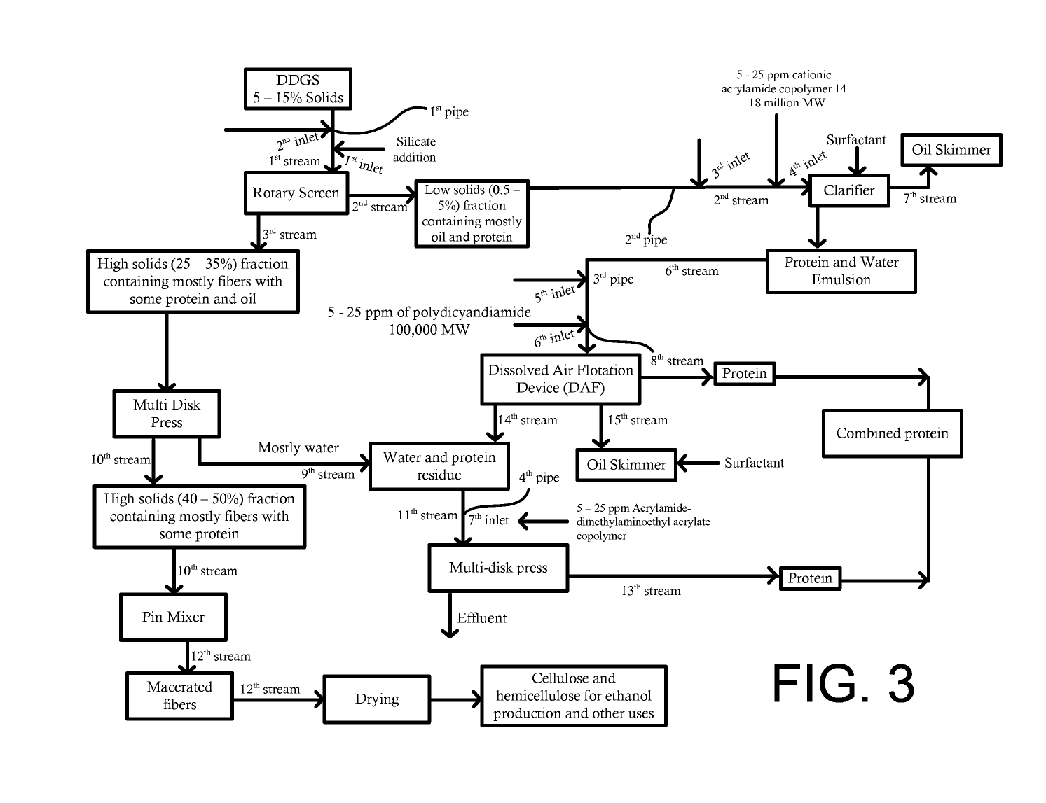

The process of the present invention is described in FIGS. 1-3. FIGS. 1 and 2 represent a process used for separating a DDGS stream having a pH of about 5 or higher. FIG. 3 represents a process used for separating a DDGS stream having a pH below about 5.

In the embodiment displayed in FIG. 1, a source stream containing predominantly Dried Distillers Grains with Solubles (DDGS) stream, labeled as the 1^{st} stream, containing between about 5% to about 15% solids is fed through a 1^{st} pipe that contains a first chemical additive inlet and a second chemical additive inlet set about 15 seconds apart calculated based on the average volumetric flow rate through the pipe. About 5 ppm to about 25 ppm of a cationic polyamine having a weight average MW of about 800,000 and about a 50% charge are added to the first inlet and about 5 ppm to about 25 ppm of an anionic acrylamide copolymer having a weight average MW of between about 14,000,000 to about 22,000,000 is added to the second inlet. The DDGS stream is passed through a rotary screen where it is split into a relatively high solids stream shown as the 3^{rd} stream and a low solids stream shown as the 2^{nd} stream. The 3^{rd} stream has a percent solids content of between about 25% to about 35% solids and contains mostly cellulose and hemicellulose fibers but may also contain between about 10% to about 15% protein and typically less than 5% oil. The second stream contains about 0.5% to 10% solids in the form of protein and oil.

In another embodiment of the present invention, the process may optionally further comprise adding between about 5 ppm to about 25 ppm of a silicate to a silicate addition inlet placed in the first chemical additive pipe. The silicate inlet precedes the first inlet in the first chemical additive pipe. The silicate may be sodium silicate, calcium silicate, magnesium silicate, potassium silicate or silicon dioxide.

The third stream undergoes a further split in a multi-disk press that generates a 10^{th} stream that has a percent solids content of between about 45% to about 55% solids and contains mostly cellulose and hemicellulose fibers and a 9^{th} stream containing mostly water with small amounts of protein that might still be present that may range from about 2% to about 10%.

The second stream is moved toward a clarifier through the 2^{nd} pipe that has the 3^{rd} and 4^{th} chemical addition inlets. The 4^{th} inlet is placed about 15 seconds after the 3^{rd} inlet calculated based on the average volumetric flow rate through the 2^{nd} pipe. About 5 to about 25 ppm of cationic polyamine, 50% charge and 1 million MW is fed into the 3^{rd} inlet and 5-25 ppm anionic acrylamide copolymer 14-22 million MW are fed into the 4^{th} inlet. The clarifier splits the 4^{th} stream into the 6^{th} stream containing predominantly protein and water and the 7^{th} stream that contains predominantly oil. A skimmer may be used to collect the oil from the top of the clarifier as will be described below. The sixth stream is fed into a Dissolved Air Floatation Device (DAF) through the third pipe that contains the 5^{th} and 6^{th} chemical addition inlets prior to the DAF. The 6^{th} inlet is placed about 15 seconds after the 5^{th} inlet calculated based on the average volumetric flow rate through the 3^{rd} pipe. About 5 to 25 ppm of polydicyandiamide having about 100,000 weight average MW and having a cationic charge are added to the 5^{th} inlet and about 5-25 ppm anionic acrylamide copolymer having a weight average MW of 18-25 million are added at the 6^{th} inlet. All ppm are calculated on a weight basis.

The DAF separates the 6^{th} stream into an 8^{th} stream rich in protein with >70% of the stream composition and a water and residue stream shown as the le stream containing predominantly water and less than 15% protein. Streams 9 and 14 may be combined as they have similar compositions. The combination of these two streams forms stream number 11. The 11^{th} stream is treated in a multi disk press with about 5-25 ppm acrylamide-dimethylaminoethyl acrylate copolymer (ADMAEA) added to the 7^{th} inlet at the multidisc press or in a pipe prior to the multi-disk press. This stage further separates a 13^{th} stream containing over 80% protein from the 11^{th} stream that also generates an effluent stream comprising of mostly water. Any residual oil present in the 6^{th} stream may be collected as it rises to the top of the DAF using an oil skimmer.

In this embodiment of the present invention, two steps of protein separation are performed, wherein protein is progressively removed from the liquid portion of DDGS using suitable polymers in each stage configured for coalescing protein molecules and remove from a source stream. The 8^{th} and 13^{th} streams are rich in protein in excess of 75% by weight and may be combined into one stream, then taken for further processing as needed in order to utilize the materials.

The 10^{th} stream containing cellulose and hemicellulose fibers at solids exceeding 45% by weight may further be treated in a pin macerator according to the process disclosed in U.S. Pat. No. 8,444,810. This treatment macerates the fibers in a way that allows further extraction of ethanol-biofuels and other chemicals in subsequent steps. Typical addition levels for the polymers used in this process is between 5 ppm to about 25 ppm and preferable between about 10 ppm to about 20 ppm, the DAF must be equipped with micronized air as essential part of the DAF system. Suitable equipment used in this process, i.e., the centrifuge, clarifier and DAF may be of any type currently used in the art.

In both the clarifier and the DAF device, the oil tends to rise to the top of the solids fraction as it is lighter than the protein fraction. An oil skimmer may optionally be used to skim off the oil; oil skimmers are currently known in the art and a number of suitable skimming devices may be used for this purpose. Skimming aids may be optionally added to help with agglomerating the oil to facilitate it being skimmed off. Sulfonic acids such as Nonylphenol, (ethoxylated) ethanol acid with the chemical formula of or sodium dodecylbenzenesulfonate having the formula C15H24O.(C2H4O)n are surfactants that are suitable for this purpose. Likewise, silicon dioxide, SiO₂, is suitable as an oil skimming aid. These aids may be added with the polymers.

The embodiment presented in FIG. 2 includes a centrifuge into which the 2^{nd} stream is fed. The centrifuge splits the second stream into a 4^{th} stream and a 5^{th} stream. The 4^{th} stream is moved toward a clarifier through the 2^{nd} pipe while the 5^{th} stream is passed toward the Dissolved Air Floatation Device (DAF) through the third pipe. The second pipe has the third and fourth chemical addition inlets. The 4^{th} inlet is placed about 15 seconds after the 3^{rd} inlet calculated based on the average volumetric flow rate through the 2^{nd} pipe. The 6^{th} inlet is placed about 15 seconds after the 5^{th} inlet calculated based on the average volumetric flow rate through the 3^{rd} pipe. Prior to passing through the clarifier, the 4^{th} stream is treated with about 5 ppm to about 25 ppm of a cationic polyamine having a weight average MW of about 1,000,000 and about a 50% charge. About 5 to 25 ppm of polydicyandiamide having about 100,000 weight average MW and having a cationic charge are added to the 5^{th} inlet and about 5-25 ppm anionic acrylamide copolymer having a weight average MW of 18-25 million are added at the 6^{th} inlet.

The clarifier separates the 4^{th} stream into a 6^{th} stream rich in protein with >70% of the stream composition, and a 7^{th} stream rich in oil with >70% content of the composition. The 4^{th} stream may contain between about 10% to about 20% protein while the fifth stream contains over 90% water.

With both embodiments illustrated in FIGS. 1 and 2, a silicate may optionally be added to the first pipe prior to the rotary screen. The silicate may be sodium silicate, potassium silicate, magnesium silicate, silicon dioxide or calcium silicate.

In the process described in FIG. 3, a silicate is added into the first chemical addition inlet in lieu of the cationic polyamine and anionic acrylamide copolymer. No chemical is added into the third chemical addition inlet or the sixth chemical addition inlet, while a cationic acrylamide copolymer is added to the 4^{th} chemical addition inlet. The other process step are similar to those used with DDGS having a pH of 5 or above.

It should be understood, of course, that the foregoing relates to exemplary embodiments of the invention and that modifications may be made without departing from the spirit and scope of the invention.

I claim:

1. A multi-stage substantially continuous process for separating a source stream having a pH above 5.0, said stream intermixedly containing fibers, proteins and oil, said process being configured for separating the source stream into three streams each containing predominantly one component, said source stream containing dried distillers grains with solubles, said process comprising the stages of:
providing a first stream comprising dried distillers grain with solubles, said dried distillers grain stream containing water, oil, protein and fibers, said fibers containing hemicellulose and cellulose components;
separating from the source stream a stream comprising predominantly proteins and oil in a water mixture form and a stream comprising predominantly fiber materials;
separating a stream containing predominantly oil and a stream containing predominantly proteins from the water mixture;
progressively concentrating the stream containing predominantly proteins in at least one additional step.

2. The process of claim 1, wherein separating a stream comprising predominantly proteins and oil in a water mixture form and a stream comprising predominantly fiber materials from the source stream comprises:
passing said source stream through a first chemical additive pipe having a first chemical addition inlet and a second chemical addition inlet, said first chemical additive pipe leading toward a rotary screen, said second chemical addition configured to occur about 15 seconds after the first chemical addition based on an average volumetric flow rate through the pipe;
adding between about 5 to about 25 ppm of a first cationic polyamine to the first stream at said first chemical addition inlet and a first anionic acrylamide copolymer to the first stream at the second outlet; and
separating a second stream and a third stream from said first stream in the rotary screen, said second stream containing predominantly oil and protein in a non-aqueous fluid form, said third stream containing predominantly cellulose and hemicellulose fibers.

3. The process of claim 1, wherein separating a stream containing predominantly oil and a stream containing predominantly proteins from the water and oil mixture comprises:
passing said second stream through a second chemical additive pipe having a third chemical addition inlet and a fourth chemical addition inlet, said second chemical additive pipe leading toward a clarifier, said fourth chemical addition configured to occur about 15 seconds after the third chemical addition based on an average volumetric flow rate through the pipe;
adding between about 5 to about 25 ppm of a second cationic polyamine to said third chemical addition inlet and adding between about 5 to about 25 ppm of a second anionic acrylamide copolymer to the fourth chemical addition inlet;
feeding said second stream into the clarifier wherein actions of said clarifier separate said second stream into a sixth stream and a seventh stream, said sixth stream containing predominantly a protein mixture and said seventh stream containing predominantly oil;
passing said sixth stream through a third chemical additive pipe having a fifth chemical addition inlet and a sixth chemical addition inlet, said third chemical additive pipe leading toward a dissolved air floatation device, said sixth chemical addition configured to occur about 15 seconds after the fifth chemical addition;
adding between about 5 to about 25 ppm of polydicyandiamide to said fifth chemical addition inlet and adding between about 5 to about 25 ppm of a third anionic acrylamide copolymer to the sixth outlet; and
feeding said sixth stream into a dissolved air floatation device wherein actions of said dissolved air floatation device separate an eighth stream and a fourteenth stream from said sixth stream, said eighth stream containing predominantly protein, said fourteenth stream containing predominantly a water and protein mixture.

4. The process of claim 3, further comprising adding between about 5 to about 25 ppm of a surfactant to the second stream to aid in removing the oil from the clarifier, said surfactant being selected from the group consisting of sulfonic acid and silicon dioxide, said oil being removed by an oil skimmer.

5. The process of claim 4, further comprising adding between about 5 to about 25 ppm of a surfactant to the sixth stream or the fifteenth stream to aid in removing the oil from the dissolved air floatation device, said surfactant being selected from the group consisting of sulfonic acid and silicon dioxide, said oil being removed by an oil skimmer.

6. The process of claim 5, further comprising passing said third stream through a multi-disk press wherein actions of said multi-disk press separate a tenth stream and an ninth stream from said third stream, said tenth stream containing predominantly cellulose and hemicellulose fibers, said ninth stream containing predominantly water and protein.

7. The process of claim 6 further comprising macerating the fibers contained in the tenth stream in a pin mixer according to an embodiment described in U.S. Pat. No. 8,444,810 to produce a twelfth stream containing macerated fibers.

8. The process of claim 7 further comprising combining the ninth stream and the fourteenth stream to form an eleventh stream and passing said eleventh stream into a multi disk press and further comprising adding to said multi-disc press between about 5 ppm to about 25 ppm on a weight basis of acrylamide-dimethylaminoethyl acrylate copolymer in a manner as to separate the eleventh stream into a thirteenth stream comprising predominantly protein and into an effluent stream containing predominantly water.

9. The process of claim 8 further comprising combining the thirteenth stream and the eighth stream in order to consolidate streams containing predominantly protein.

10. The process of claim 9 further comprising drying the macerated fibers contained in the twelfth stream.

11. The process of claim 10, further comprising a centrifuge for receiving and processing the second stream wherein said centrifuge separates a fourth stream and a fifth stream from said second stream, said fourth stream containing predominantly an oil and protein mixture, said fourth stream being directed toward the clarifier and said fifth stream being directed toward the dissolved air floatation device.

12. The process of claim 2 further comprising adding between about 5 ppm to about 25 ppm on a weight basis of a silicate compound to a silicate addition inlet, said silicate addition inlet being disposed on the first chemical additive pipe and preceding the first chemical addition inlet, said silicate compound being selected from the group consisting of silicon dioxide, magnesium silicate, calcium silicate, sodium silicate and potassium silicate.

13. A multi-stage substantially continuous process for separating a source stream having a pH below 5.0, said stream intermixedly containing fibers, proteins and oil, said process being configured for separating the source stream into three streams each containing predominantly one component, said source stream containing dried distillers grains with solubles, said process comprising the stages of:
providing a first stream comprising dried distillers grain with solubles, said dried distillers grain stream containing water, oil, protein and fibers, said fibers containing hemicellulose and cellulose components;
separating from the source stream a stream comprising predominantly proteins and oil in a water mixture form and a stream comprising predominantly fiber materials;
separating a stream containing predominantly oil and a stream containing predominantly proteins from the water mixture;
progressively concentrating the stream containing predominantly proteins in at least one additional step.

14. The process of claim 13, wherein separating a stream comprising predominantly proteins and oil in a water mixture form and a stream comprising predominantly fiber materials from the source stream comprises:
passing said source stream through a first chemical additive pipe having a first chemical addition inlet and a second chemical addition inlet, said first chemical additive pipe leading toward a rotary screen, said second chemical addition configured to occur about 15 seconds after the first chemical addition based on an average volumetric flow rate through the pipe;
adding between about 5 to about 25 ppm of a silicate to the first stream at said first chemical addition inlet, said silicate being selected from the group consisting of sodium silicate, potassium silicate, magnesium silicate, silicon dioxide and calcium silicate; and
separating a second stream and a third stream from said first stream in the rotary screen, said second stream containing predominantly oil and protein in a non-aqueous fluid form, said third stream containing predominantly cellulose and hemicellulose fibers.

15. The process of claim 14, wherein separating a stream containing predominantly oil and a stream containing predominantly proteins from the water and oil mixture comprises:
passing said second stream through a second chemical additive pipe having a third chemical addition inlet and a fourth chemical addition inlet, said second chemical additive pipe leading toward a clarifier, said fourth chemical addition configured to occur about 15 seconds after the third chemical addition based on an average volumetric flow rate through the pipe;
adding between about 5 to about 25 ppm of a cationic acrylamide copolymer to the fourth chemical addition inlet;
feeding said second stream into the clarifier wherein actions of said clarifier separate said second stream into a sixth stream and a seventh stream, said sixth stream containing predominantly a protein mixture and said seventh stream containing predominantly oil;
passing said sixth stream through a third chemical additive pipe having a fifth chemical addition inlet and a sixth chemical addition inlet, said third chemical additive pipe leading toward a dissolved air floatation device, said sixth chemical addition configured to occur about 15 seconds after the fifth chemical addition;
adding between about 5 to about 25 ppm of polydicyandiamide to said sixth chemical addition inlet; and
feeding said sixth stream into a dissolved air floatation device wherein actions of said dissolved air floatation device separate an eighth stream and a fourteenth stream from said sixth stream, said eighth stream containing predominantly protein, said fourteenth stream containing predominantly a water and protein mixture.

16. The process of claim 15, further comprising adding between about 5 to about 25 ppm of a surfactant to the second stream to aid in removing the oil from the clarifier, said surfactant being selected from the group consisting of sulfonic acid and silicon dioxide, said oil being removed by an oil skimmer.

17. The process of claim 16, further comprising adding between about 5 to about 25 ppm of a surfactant to the sixth stream or the fifteenth stream to aid in removing the oil from the dissolved air floatation device, said surfactant being selected from the group consisting of sulfonic acid and silicon dioxide, said oil being removed by an oil skimmer.

18. The process of claim 17, further comprising passing said third stream through a multi-disk press wherein actions of said multi-disk press separate a tenth stream and an ninth stream from said third stream, said tenth stream containing predominantly cellulose and hemicellulose fibers, said ninth stream containing predominantly water and protein.

19. The process of claim 18 further comprising macerating the fibers contained in the tenth stream in a pin mixer according to an embodiment described in U.S. Pat. No. 8,444,810 to produce a twelfth stream containing macerated fibers, then drying the macerated fibers contained in the twelfth stream.

20. The process of claim 19 further comprising combining the ninth stream and the fourteenth stream to form an eleventh stream and passing said eleventh stream into a multi disk press and further comprising adding to said multi-disc press between about 5 ppm to about 25 ppm on a weight basis of acrylamide-dimethylaminoethyl acrylate copolymer in a manner as to separate the eleventh stream into a thirteenth stream comprising predominantly protein and into an effluent stream containing predominantly water, then combining the thirteenth stream and the eighth stream in order to consolidate streams containing predominantly protein.
